(19) United States
(12) Patent Application Publication
(10) Pub. No.: US 2002/0052411 A1
(43) Pub. Date: May 2, 2002

(54) Valproate compositions and processes for making

(76) Inventors: Dagmar Gobel, Radebeul (DE); Michael Pieroth, Weinbohla (DE); Bernd Terhaag, Pratzschwitz (DE); Ramona Dieckmann, Laussnitz (DE); Evelyn Heuberger-Wieland, Koln (DE)

Correspondence Address: Gabriel P. Katona L.L.P., 14th Floor, 708 Third Avenue, New York, NY, 10017, US

(21) Appl. No.: 09/794,318

(22) Filed: Feb. 27, 2001

(30) Foreign Application Priority Data
Sep. 3, 1998 (DE) 198 40 152.3

(51) Int. Cl. A61K 31/19; A61K 9/22

(52) U.S. Cl. 514/557; 424/468

(57) ABSTRACT

The invention is a delayed release pharmaceutical composition which comprises calcium valproate, one or more acrylic polymers, and on or more sugar esters, a process for making, and the treatment of epilepsy, migraine, and manic-depression therewith.

### FIELD OF INVENTION

The present invention relates to novel pharmaceutical compositions comprising calcium valproate with delayed release of active compound, in particular in tablet form, processes for their preparation and for their use for the treatment of epilepsy, migraine and manic-depressive conditions.

### BACKGROUND

Calcium valproate (calcium salt of di-n-propylacetic acid) is a known antiepileptic which is usually taken orally in a dosage of from 900 mg to 2700 mg daily when used in humans in an immediate-release form. Calcium valproate can also be used for the treatment of migraine and manic-depressive conditions. The active constituent of calcium valproate is free valproic acid.

The known calcium valproate preparations have the disadvantage that upon administration of high doses toxic blood level values occur in the influx phase, which can cause, for example, liver damage.

The release of the active compound should thus take place so that the plasma levels necessary for the therapy are achieved without intermediate occurrence of toxic blood concentrations. Furthermore, the dosage should contain an amount of calcium valproate through which the therapeutic plasma levels can be achieved and maintained by taking it one to twice daily and thus the compliance of the patient is better assured. A suitable delayed-release formulation can have a calcium valproate content of up to 900 mg or more, suitably from 250 to 600 mg, per dose.

The time of passage of a substance in the gastrointestinal tract between oral administration and entry into the large intestine can take up to 14 hours. A time of passage of 1 hour to up to 6 hours is observed for the stomach, while the time of passage in the small intestine is between 1.5 and 8 hours. In particular in the case of non-disintegrating particles of over 3 mm, such as a 10 tablet, a longer residence time and a longer time of passage can be expected of up to a total of 8 hours in the stomach. It has been shown in practice, that the complete release of the active compound should advantageously take place after 12 to 16 hours, while longer delayed release times have the disadvantage that unabsorbed active compound is excreted through the stool.

It is desired that a delayed release form should release the valproic acid active compound with a delay in the stomach at a pH, of between 1.0 and 3.0 and in the small intestine at pH 6.8 so that a uniformly delayed invasion in vivo is assured.

It is important for high dose valproic acid preparations to avoid excessively high blood levels, and this effect can only be achieved by delayed release.

A suitable release role of the active compound in vitro in a pH 6.8 buffer is, for example, represented by the following active compound release profile:

| | |
|---|---|
| after 1 hour | 15-35% release |
| after 4 hours | 50-70% release |
| after 8 hours | > 70% release |
| after 12 hours | complete release |

The percentages here in each case are based on the actual (stated) content of calcium valproate in the individual tablet. The above mentioned release profile is only one example, without intending to exclude other suitable release profiles.

Suitably the dosage form should be divisible, the parts being intended to have a comparable active compound release profile to the undivided preparation.

European patent application No. EP-A 0 230 332 describes a delayed release of active compound for the active compound ibuprofen using sucrose monopalmitate, polyvinylpyrrolidone (PVP) and stearic acid. In this case, a very high proportion of sucrose monopalmitate of 25% by weight, based on active compound is used. Such a high proportion is disadvantageous in the production of preparations with a high content of active compound because of the resulting total weight.

European patent application No. EP-A 0 385 846 describes the preparation of a delayed release tablet of comprising sodium valproate and valproic acid as active compound using highly viscous hydroxypropylmethylcellulose as the release-delaying agent, which tablet was then coated with a film of polyacrylate or methacrylate agents such as are sold under the trademark Eudragit. Film-coating has the fundamental disadvantage that the original action of the film is impaired or even lost upon division of the tablet.

European patent application No. EP-A 0 133 110 describes the use of Eudragit in combination with ethylcellulose as a release-delaying agent for a delayed-release tablet of sodium valproate and valproic acid. Ethylcellulose is employed in this case as a solution in an organic solvent, such as acetone or ethanol. The use of organic solvents, however, is disadvantageous because of their flammability and toxicity for large-scale pharmaceutical manufacturing. The high proportion of Eudragit of at least 10% by weight, based on the active compound, is also unfavourable for highly concentrated delayed-release preparations.

### BRIEF DESCRIPTION OF THE DRAWING

The present invention is described with reference being had to the drawing, wherein

FIGS. 1a and b show a tablet divisible into two parts in plan and side views, respectively;

FIGS. 2a and b show a tablet divisible into four parts in plan and side views, respectively; and

FIG. 3 shows an example of the in vitro release rate of the active ingredient and of controls, at pH 68.

Japanese patent application No. JP-86/248 211 describes the use of sucrose fatty acid esters or other surface-active excipients for the production of a pharmaceutical formulation comprising calcium valproate, 0.9% by weight of sugar ester, based on the calcium valproate, resulting in a complete dissolution of a 224 mg calcium valproate tablet within 27-46 min at pH 1. In contrast, the control tablet produced without sugar ester only dissolved after 120 min.

German patent application No. DD-A 295 543 describes the use of 3 parts of calcium valproate, 1 part of fully hydrolyzed polyvinyl alcohol and 2 parts of partly hydrolyzed polyvinyl alcohol for the production of delayed-release preparations, the degree of delay intended to depend on the ratio between fully and partly hydrolyzed polyvinyl alcohol. The disadvantage of this composition is on the one hand the high proportion of excipient, since this in the case of a highly concentrated pharmaceutical form leads to an extremely high overall mass of the oral pharmaceutical form. On the other hand, the polyvinyl alcohol used as an excipient can only be obtained with difficulty in the degree of purity demanded for pharmaceutical production.

### DESCRIPTION OF THE INVENTION

The object of the present invention is the provision of a pharmaceutical composition comprising calcium valproate with delayed release of active compound, which can have a content of up to 900 mg or even more, such as 1000 mg of calcium valproate, and a process for its reparation. The delayed-release ready-to-use preparation according to the present invention should in this case have a total or gross weight which is as low as possible and be suitably divisible.

The object according to the present invention is achieved by the use of the combination of at least one acrylic polymer and one sugar ester as a release-delaying agent.

As used in the disclosure and the claims, calcium valproate means all calcium salts of valproic acid and their complexes with valproic acid, water or other solvents.

The preparation of calcium valproate is described, for example, in German patent Nos. DD 215,530; DD 215,531; DD 215,533; and DD 293,053.

Suitable complexes are, for example, the dimer of one molecule of valproic acid and ½ mol of calcium valproate, as described in European patent application No. EP-A 34 172; polymeric calcium salts of valproic acid, monomers in which four valproic acid radicals in each case are associated with a calcium ion (see EP-A 141 267), or the complex of one mole of calcium valproate and 3 mol of valproic acid (see EP-A 282 834).

The calcium bis(di-n-propyl acetate) which is one mole of calcium and 2 mols of valproic acid is most suitable. Calcium valproate can be present under normal atmospheric pressure either as a dihydrate (approximately 9.9% water), ⅓ hydrate (approximately 3.5-3.7% water) or ¼ hydrate (approximately 2.2% water). Lower water content of crystallization can also be obtained under reduced pressure. In addition, water can also be present in a form adsorptively bound to the surface.

Calcium valproate hydrates are suitable with the calcium valproate dihydrate and calcium valproate with a water content of 3.6% by weight being particularly suitable. The dihydrate is suitably employed as a starting material in the preparation. The ⅓ hydrate (about 3.6% proportion of water) is suitably present in the final product after the drying step.

Suitable acrylic polymers of the Eudragit type according to the present invention are described in detail in the publication Handbook of Pharmaceutical Excipients, Ed.: Ainley Wade and Paul J. Weber, in the chapter “Polymethacrylates”, 2nd Edition, The Pharmaceutical Press, London, 1994, pages 362-365 and in Herbert P. Fiedler, Lexikon der Hilfsstoffe für Pharmazie, Kosmetik und angrenzende Gebiete [Encyclopedia of Excipients for Pharmacy, Cosmetics and Related Areas], Volume 1, (Volume 9 of the series “Der pharmazeutische Betrieb” [Pharmaceutical Operation], published by Cantor Aulendorf, 3rd Edition, 1989, pages 486-487.

Suitable acrylic polymers are the copolymer of ethyl acrylate and methyl methacrylate in the ratio 2:1 as are sold under the trademark Eudragit NE, the copolymer of ethyl acrylate and methyl methacrylate having a low content of quaternary ammonium groups in the ratio, 1.0:2.0:0.1 sold under the trademark Eudragit RS, and the copolymer of methacrylic acid and ethyl acrylate in the ratio 1:1 sold under the trademark Eudragit L, and in the ratio 1:2 sold under the trademark Eudragit S. The copolymer ratios mentioned are particularly advantageous. However, copolymers having other ratios or the polymers polyethyl acrylate, polymethyl methacrylate and polymethacrylic acid can also be used. These terms as defined above are employed throughout the disclosure and the claims.

Eudragit NE, Eudragit L and mixtures thereof are particularly suitable.

It is also possible to employ Eudragit NE and shellac, the latter suitably in aqueous solution, such as sold, for example, by Marchand & Chie GmbH, Mainz, Germany.

The sugar esters according to the present invention are characterized in that they have an HLB (hydrophilic/lipophilic balance) value greater than 10, suitably between 14 and 16, particularly suitably of 15 and in water at 25° C. have a solubility of no more than 1 part of ester to 100 parts of water (parts by weight), and at 60° C. to 80° C. a solubility of at least 1 part of ester to 10 parts of water (parts by weight). The HLB value is a numerical system which describes the hydrophilicity/lipophilicity equilibrium of surface-active substances. For the determination of the HLB, see, for example, Herbert Stricker (Ed.) in “Physikalische Pharmazie”(Physical Pharmacy], 3rd Edition, pages 96 and 97, 1987.

Suitable sugar esters are, for example, sucrose palmitate or sucrose stearate.

A suitable sugar ester is sucrose palmitate having a content of about 70% of monopalmitate. Sucrose palmitate having a content of about 70% of monopalmitate and about 20% of dipalmitate and about 10% of tripalmitate is, for example, sold by Mitsubishi, Japan.

With respect to the structure and the known properties of the sugar esters, reference is made to Herbert P. Fiedler, Lexikon der Hilfsstoffe frir Pharmazie, Kosmetik und angrenzende Gebiete [Encyclopedia of Excipients for Pharmacy, Cosmetics and Related Areas], Volume 2, (Volume 9 of the series “Der pharmazeutische Betriebm [Pharmaceutical Operation), published by Cantor Aulendorf, 3rd Edition, 1989, pages 1064-1067.

The composition according to the invention can suitably also contain an antiadhesive agent, such as talcum.

Suitable excipients which can also be contained are a lubricant, such as magnesium stearate, stearic acid or sodium stearyl fumarate, and/or a filler, such as microcrystalline cellulose or lactose monohydrate.

The calcium valproate content of the composition according to the present invention can be between 150 and 900 mg or even above this limit, for example 1000 mg, suitably between 250 and 600 mg, per dosage unit.

It has been surprisingly found that the desired delayed-release effects are achieved even with very low contents of polyacrylate such as of 2 to 8% by weight, based on active compound.

According to a suitable embodiment of the present invention, it has been found that an Eudragit NE content in the range from, 1 to 5% by weight, preferably 2 to 3% by weight, particularly suitably about 2% by weight, and a Eudragit L content of 1 to 3% by weight, suitably 2 to 3% by weight, particularly suitably about 2.5% by weight, in each case based on the active, are advantageous for the desired delayed release profiles.

The sugar ester content can be in the range of from 0.9 to 10% by weight, suitably 4 to 6% by weight, particularly suitably about 5% by weight, based on the active.

According to a suitable embodiment, a pharmaceutical composition according to the present invention contains from 150 to 1000 mg of calcium valproate, from 1 to 5% by weight, suitably from 2 to 3% by weight, particularly suitably about 2% by weight Eudragit NE; from 1 to 3% by weight, suitably 2 to 3% by weight, particularly suitably about 2.5% by weight of Eudragit L; and from 0. 9 to 10% by weight, suitably from 4 to 6% by weight, particularly suitably about 5% by weight sucrose palmitate; the percentages in each case being based on the calcium valproate active.

The polyacrylates can be employed as an aqueous dispersion, suitably as a 30% strength by weight dispersion in water.

The aqueous solution of the sugar ester can have a sugar ester content of from 10 to 20% by weight at a water temperature of from 60° C. to 80° C. The sugar esters can be employed as an aqueous solution, with a 10 to 20% by weight solution being suitable.

The sucrose palmitate suitably used as the sugar ester has up to 70% of the monoester and dissolves at a temperature of 70° C. in water at a concentration of from 10 to 20% by weight of sucrose palmitate, while at 25° C. it is only poorly soluble in water.

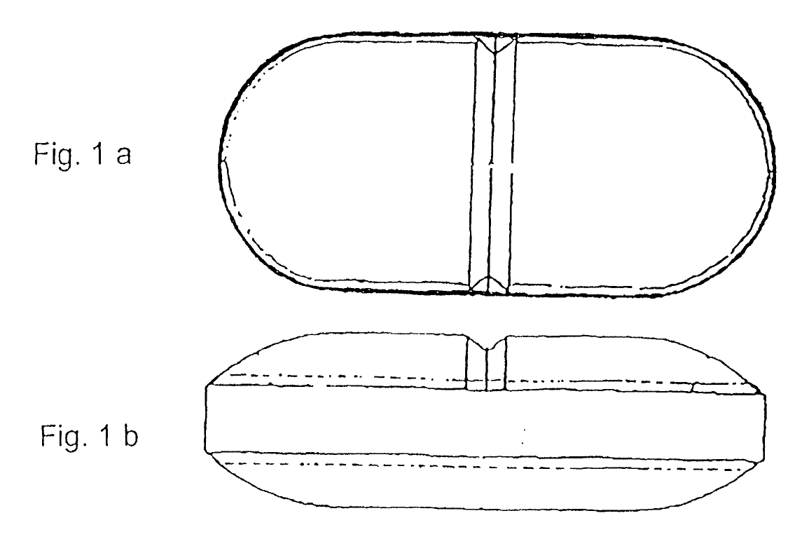

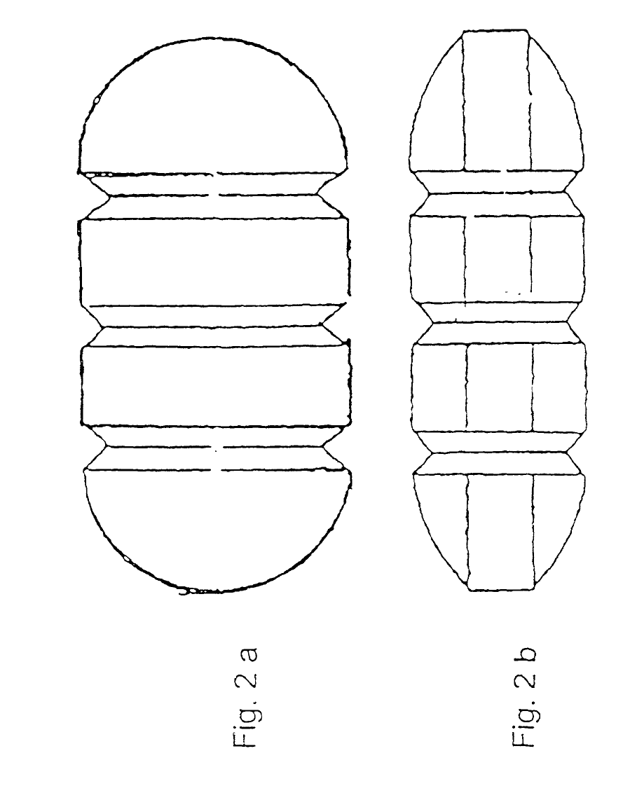

In a suitable embodiment, the pharmaceutical preparation of calcium valproate according to the present invention with delayed release of active compound is a tablet having an active compound content of 600 mg. The tablet can have an oblong shape and, as shown in FIGS. 1a and 1b, additionally a notch for division into two, or, as shown in FIGS. 2a and 2b, having three encircling notches for division into four parts.

Instead of this or in addition, it can also have other shapes and recesses for achieving divisibility (not shown).

The tablet can be provided with a film coating which can contain suitable flavorings or colorants.

The pharmaceutical preparation according to the present invention exhibits the in-vitro release rate that is suitable from the medicinal point of view.

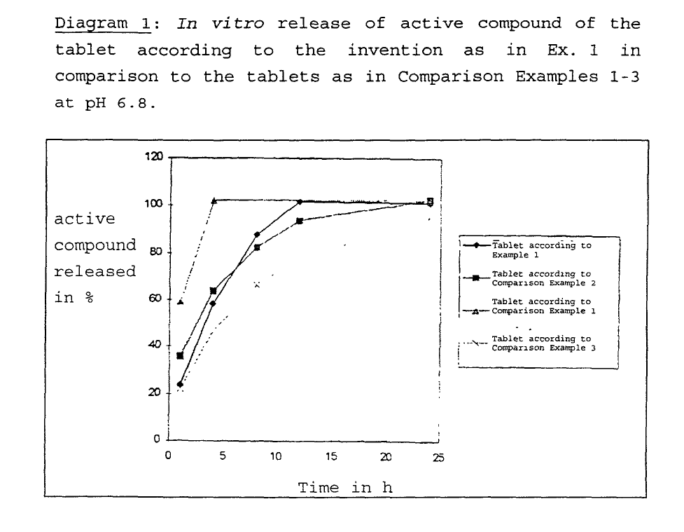

The release rate shown in FIG. 3 evidences the unobviousness of the present invention to a person skilled in the art, because with the mere admixture of, for example, 2 to 4.5% by weight of acrylic polymers without sugar ester only an incomplete release is obtained after 12-16 hours, whereas the sugar ester on its own causes no noticeable delay in the release.

When using the combination according to the present invention of, for example, 5% by weight of sugar ester, which for its part only causes a maximal delay of the release of calcium valproate of 4 hours, a control of the release rate profile now occurs within the period of from 4-12 to 4-16 hours, with for example 50-70% of the release occurring after a total of 4 hours, and complete release after at most 12-16 hours, attesting to the uniqueness of result of the combination according to the present invention of acrylic polymer and sugar ester for delaying the release of valproic acid.

The pharmaceutical preparation according to the present invention can be administered by taking orally once or more often daily. According to this dosage scheme according to the present invention, the treatment of epilepsy, migraine and manic-depressive conditions is conveniently possible.

According to a further feature of the present invention, a process for the preparation of a pharmaceutical composition comprising calcium valproate with delayed release of active compound is provided comprising the steps of admixing calcium valproate and an aqueous dispersion of Eudragit NE, or Eudragit RS, or mixtures thereof, or an aqueous dispersion comprising Eudragit L or Eudragit S or shellac or mixtures thereof, and an aqueous solution of a sugar ester, optionally with suitable excipients, such as talc, microcrystalline cellulose and magnesium stearate, and with one or more drying steps to obtain a tabletting mixture, and compressing the mixture into tablets.

In a suitable embodiment the aqueous dispersion of Eudragit L and/or Eudragit S, or shellac or mixtures thereof also contains talc.

In a suitable embodiment, the admixture takes place in the sequence 1.) Eudragit NE or Eudragit RS or mixtures thereof, 2.) Eudragit L or Eudragit S or shellac or mixtures thereof and 3.) sugar esters, in each case followed by a drying step.

The admixture, however, can also take place in a different sequence.

It has been surprisingly found that the production of the pharmaceutical preparation according to the present invention is possible when using aqueous sugar ester solutions. The aqueous sugar ester solution can be added as a warmed or hot solution. Suitably, the addition of the aqueous sugar ester solution should be carried out so that the water is completely or partially evaporated. This can be carried out, for example, in a suitable fluidized-bed drying device or in an aerated stirring device.

The pharmaceutical compositions according to the present invention can be used for the treatment of epilepsy (in particular for the prophylaxis of Grand mal, myoclonic attacks and absences in primary generalized epilepsies), manic-depressive conditions and migraine in mammals, in particular in humans.

The term “treatment” as used herein is intended to include all measures in the course of therapy and/or prophylaxis of the disease.

The term “mammals” as used herein in particular includes humans in all stages of age and development and of both sexes, and also domestic animals, such as dogs and cats, and agricultural animals, such as horses, cows, sheep and goats.

“Epilepsy” as used herein is to be understood as including all types of attacks belonging to this syndrome. All forms and degrees of severity of migraine and of the conditions of the manic-depressive type are treatable using the preparations according to the present invention.

The administration of the pharmaceutical preparations is carried out orally. The number and dosage of the daily administrations is dependent on the nature and the degree of severity of the condition and on the age and state of health of the patient. Thus the treatment of epilepsy can be carried out, for example, by one- to two-times daily oral administration of the composition according to the present invention.

The following examples and controls are intended to illustrate the invention in greater detail, but without restricting the invention to these examples.

### EXAMPLE 1

40.0 g of poly(ethyl acrylate/methyl methacrylate) dispersion (30% by weight) (Eudragit NE 30 D) are introduced into 666.0 g of calcium valproate dihydrate (corresponding to 600 g of calcium valproate) and then the water of the dispersion is dried off.

Following this, 50.0 g of an aqueous 30% methacrylic acid/ethyl acrylate copolymer dispersion (Eudragit L 30 D-55) and 8 g of talc suspended therein are applied and then the water of the dispersion is dried off.

As the third step, the 10% strength by weight aqueous solution of the sugar ester with 30 g of sugar ester constituent is applied and subsequently the introduced water and the water of hydration of the active compound are dried off down to 3.6% by weight, based on the proportion of active compound.

The tabletting mixture is prepared using 20.0 g of microcrystalline cellulose and 3.0 g of magnesium stearate. It is then compressed to give a divisible tablet.

The composition of the tablet obtained:

| | | |
|---|---|---|
| calcium valproate | 600 | mg |
| poly(ethyl acrylate/methyl methacrylate) | 12 | mg |
| (Eudragit NE) | | |
| poly(methacrylic acid/ethyl acrylate) | 15 | mg |
| (Eudragit L) | | |
| talc | 8 | mg |
| sucrose palmitate | 30 | mg |
| microcrystalline cellulose | 20 | mg |
| magnesium stearate | 3 | mg |

### EXAMPLE 2

50.0 g of 30% poly(ethyl acrylate/methyl methacrylate) dispersion (Eudragit NE 30 D) are introduced into 666.0 g of calcium valproate dihydrate and then the water of the dispersion is dried off.

Following this, 50.0 g of 30% methacrylic acid/ethyl acrylate copolymer dispersion (Eudragit L 3b D-55) and 8 g of talc suspended therein are applied and then the water of the dispersion is dried off.

As the third step, the 10% strength by weight aqueous solution of the sugar ester with 30 g of sugar ester constituent is applied and subsequently the introduced water and the water of hydration of the active compound are dried off down to a proportion of 3.6% by weight.

The tabletting mixture is prepared using 20.0 g of microcrystalline cellulose and 3.0 g of magnesium stearate. It is then compressed to give a divisible tablet.

The composition of the tablet obtained:

| | | |
|---|---|---|
| calcium valproate | 600 | mg |
| poly(ethyl acrylate/methyl methacrylate) | 15 | mg |
| poly(methacrylic acid/ethyl acrylate) | 15 | mg |
| talc | 8 | mg |
| sucrose palmitate | 30 | mg |
| microcrystalline cellulose | 20 | mg |
| magnesium stearate | 3 | mg |

### EXAMPLE 3

34.09 of 30% poly(ethyl acrylate/methyl methacrylate) dispersion (Eudragit NE 30 D) are introduced into 666.0 g of calcium valproate dihydrate and then the water of the dispersion is dried off.

Following this, 50.0 g of 30% methacrylic acid/ethyl acrylate copolymer dispersion (Eudragit L 30 D-55) and 8 g of talc suspended therein are applied and then the water of the dispersion is dried off.

As the third step, the 10% strength by weight aqueous solution of the sugar ester with 30 g of sugar ester constituent is applied and subsequently the introduced water and the water of hydration of the active compound are dried off down to a proportion of 3.6% by weight.

The tabletting mixture is prepared using 20.0 g of microcrystalline cellulose and 3.0 g of magnesium stearate. It is then compressed to give a divisible tablet.

The composition of the tablet obtained:

| | | |
|---|---|---|
| calcium valproate | 600 | mg |
| poly(ethyl acrylate/methyl methacrylate) | 10 | mg |
| (Eudragit NE) | | |
| poly(methacrylic acid/ethyl acrylate) | 15 | mg |
| (Eudragit L) | | |
| sucrose palmitate | 30 | mg |
| talc | 8 | mg |
| microcrystalline cellulose | 20 | mg |
| magnesium stearate | 3 | mg |

### EXAMPLE 4

80.0 g of 30% poly(ethyl acrylate/methyl methacrylate) dispersion (Eudragit NE 30 D) are introduced into 666.0 g of calcium valproate dihydrate and then the water of the dispersion is dried off.

Following this, 40.0 g of 30% methacrylic acidlethyl acrylate copolymer dispersion (Eudragit L 30 D-55) and 8 g of talc suspended therein are applied and then the water of the dispersion is dried off.

As the third step, the 10% strength by weight aqueous solution of the sugar ester with 30 g of sugar ester constituent is applied and subsequently the introduced water and the water of hydration of the active compound are dried off down to a proportion of 3.6% by weight.

The tabletting mixture is prepared using 20.0 g of microcrystalline cellulose and 3.0 g of magnesium stearate. It is then compressed to give a divisible tablet.

The composition of the tablet obtained:

| | | |
|---|---|---|
| calcium valproate | 600 | mg |
| poly(ethyl acrylate/methyl methacrylate) | 24 | mg |
| (Eudragit NE) | | |
| poly(methacrylic acid/ethyl acrylate) | 12 | mg |
| (Eudragit L) | | |
| talc | 8 | Mg |
| sucrose palmitate | 30 | mg |
| microcrystalline cellulose | 20 | mg |
| magnesium stearate | 3 | mg |

### EXAMPLE 5

(500 mg)

The preparation was carried out analogously to Example 1 using 555 g of calcium valproate dihydrate, 33.3 g of 30% poly(ethyl acrylate/methyl methacrylate) dispersion (Eudragit NE 30 D), 41.7 g of 30% poly(methacrylic acid/ethyl acrylate) dispersion (Eudragit L 30 D-55), 6.67 g of talc, 25 g of sucrose palmitate, and 16.67 g of microcrystalline cellulose and 2.5 g of magnesium stearate.

The composition of the tablet obtained:

| | | |
|---|---|---|
| calcium valproate | 500 | mg |
| poly(ethyl acrylate/methyl methacrylate) | 10 | mg |
| (Eudragit NE) | | |
| poly(methacrylic acid/ethyl acrylate) | 12.5 | mg |
| (Eudragit L) | | |
| sucrose palmitate | 25 | mg |
| talc | 6.67 | mg |
| microcrystalline cellulose | 16.67 | mg |
| magnesium stearate | 2.5 | mg |

### EXAMPLE 6

(300 mg)

The preparation was carried out analogously to Example 1 using 666 g of calcium valproate dihydrate, 40.0 g of 30% poly(ethyl acrylate/methyl methacrylate) dispersion (Eudragit NE 30 D), 50.0 g of 30% poly(methacrylic acid/ethyl acrylate) dispersion (Eudragit L 30 D-55), 8.0 g of talc, 30.0 g of sucrose palmitate, and 20.0 g of microcrystalline cellulose and 3.0 g of magnesium stearate.

The composition of the tablet obtained:

| | | |
|---|---|---|
| calcium valproate | 300 | mg |
| poly(ethyl acrylate/methyl methacrylate) | 6 | mg |
| (Eudragit NE) | | |
| poly(methacrylic acid/ethyl acrylate) | 7.5 | mg |
| (Eudragit L) | | |
| sucrose palmitate (sucrose ester 15) | 15 | mg |
| talc | 4 | mg |
| microcrystalline cellulose | 10 | mg |
| magnesium stearate | 1.5 | mg |

### EXAMPLE 7

(250 mg)

The preparation was carried out analogously to Example 1 using 555 g of calcium valproate dihydrate, 33.3 g of 30% poly(ethyl acrylate/methyl methacrylate) dispersion (Eudragit NE 30 D), 41.7 g of 30% poly(methacrylic acid/ethyl acrylate) dispersion (Eudragit L 30 D-55), 6.67 g of talc, 25.0 g of sucrose palmitate, and 16.67 g of microcrystalline cellulose and 2.5 g of magnesium stearate.

The composition of the tablet obtained:

| | | |
|---|---|---|
| calcium valproate | 250 | mg |
| poly(ethyl acrylate/methyl methacrylate) | 5 | mg |
| (Eudragit NE) | | |
| poly(methacrylic acid/ethyl acrylate) | 6.25 | mg |
| (Eudragit L) | | |
| sucrose palmitate | 12.5 | mg |
| talc | 3.3 | mg |
| microcrystalline cellulose | 8.3 | mg |
| magnesium stearate | 1.25 | mg |

Controls 1 to 3

Control 1: (only sugar ester)

The preparation was carried out analogously to Example 1 using 666 g of calcium valproate dihydrate, 30.0 g of sucrose palmitate, and 20.0 g of microcrystalline cellulose and 3.0 g of magnesium stearate.

| | | |
|---|---|---|
| calcium valproate | 600 | mg |
| sucrose palmitate | 30 | mg |
| microcrystalline cellulose | 20 | mg |
| magnesium stearate | 3 | mg |

The composition of the tablet obtained:

Control 2: (only poly(ethyl acrylate/methyl methacrylate))

The preparation was carried out analogously to Example 1 using 666 g of calcium valproate dihydrate, 40.0 g of 30% poly(ethyl acrylate/methyl methacrylate) dispersion (Eudragit NE 30 D), and 20.0 g of microcrystalline cellulose and 3.0 g of magnesium stearate.

The composition of the tablet obtained:

| | | |
|---|---|---|
| calcium valproate | 600 | mg |
| poly(ethyl acrylate/methyl methacrylate) | 12 | mg |
| (Eudragit NE) | | |
| microcrystalline cellulose | 20 | mg |
| magnesium stearate | 3 | mg |

Control 3: (only poly (ethyl acrylate/methyl methacrylate) and poly(methacrylic acid/ethyl acrylate))

The preparation was carried out analogously to Example 1 using 666 g of calcium valproate dihydrate, 40.0 g of 30% poly(ethyl acrylate/methyl methacrylate) dispersion (Eudragit NE 30 D), 50.0 g of 30% poly(methacrylic acid/ethyl acrylate) dispersion (Eudragit L 30 D-55), 8.0 g of talc and 20.0 g of microcrystalline cellulose and 3.0 g of magnesium 15 stearate.

The composition of the tablet obtained:

| | |
|---|---|
| calcium valproate | 600 mg |
| poly(ethyl acrylate/methyl methacrylate) | 12 mg |
| (Eudragit NE) | |
| poly(methacrylic acid/ethyl acrylate) | 15 mg |
| (Eudragit L) | |
| talc | 8 mg |
| microcrystalline cellulose | 20 mg |
| magnesium stearate | 3 mg |

Investigation of the active compound release behavior:

FIG. 3 shows the delayed release of active compound of the tablet according to the invention as in Example I in comparison to the tablets as in Controls 1 to 3. It is evident that a complete release takes place within 12 hours only when using the composition according to the present invention, comprising calcium valproate, acrylic polymer and sugar ester. In contrast, the use of only sugar ester (Control 1) leads to a rapid release within 4 hours, while the use of only poly(ethyl acrylate/methylmethacrylate) (Control 2) and poly(ethyl acrylate/methyl methacrylate) with poly(methacrylic acid/ethyl acrylate) (Control 3) leads to a complete release of active compound at pH 6.8 only within 24 hours or later.

The investigations on the active compound release behavior were carried out in a borate buffer of pH 6.8 (according to Palitzsch) using a blade stirrer apparatus according to Ph. Eur. General Methods 2.9.3. The quantitative determination of valproic acid was carried out by gas chromatography.

It was furthermore seen that the tabletting mixture according to the invention as in Example 1 can be readily compressed to give tablets. In contrast, the tabletting mixture as of Controls 2 and 3 tended to adhere the compressed tablets to the tabletting tools in spite of the presence of the antiadhesive talc in Control 3. The non-adhesion of the tabletting mixture according to the present invention represents a further considerable advantage, in particular in the case of complicated shapes such as for tablets having three encircling dividing notches for example, tablets according to FIGS. 2a and 2b).

1. Pharmaceutical composition with delayed release of active compound, characterized in that it comprises calcium valproate, at least one acrylic polymer and at least one sugar ester.

2. Pharmaceutical composition according to claim 1, characterized in that the acrylic polymer is selected from the group consisting of Eudragit® NE, Eudragit® L, Eudragit® S, Eudragit® RS and mixtures thereof.

3. Pharmaceutical composition according to claim 1 or 2, characterized in that part of the acrylic polymer is replaced by shellac.

4. Pharmaceutical composition according to one of claims 1 to 3, characterized in that the sugar ester has an HLB of greater than 10, preferably of 14 to 16, particularly preferably of 15 to 16, and a water solubility at 37° C. of no more than 1 part of sugar ester to 100 parts of water, and a water solubility at 60-80° C. of at least 1 part of sugar ester to 10 parts of water, in each case based on the weight.

5. Pharmaceutical composition according to one of claims 1 to 4, characterized in that the sugar ester is a sucrose palmitate or sucrose stearate.

6. Pharmaceutical composition according to one of claims 1 to 5, characterized in that the sugar ester is sucrose palmitate having a proportion of approximately 70% by weight of sucrose monopalmitate, based on sucrose palmitate employed.

7. Pharmaceutical composition according to one of claims 1 to 6, characterized in that it contains 150 to 1000 mg of calcium valproate, 1 to 5% by weight, preferably 2 to 3% by weight, of Eudragit® NE, particularly preferably approximately 2% by weight, 1 to 3% by weight, preferably 2 to 3% by weight, particularly preferably approximately 2.5% by weight, of Eudragit® L and 0.9 to 10% by weight, preferably 4 to 6% by weight, particularly preferably approximately 5% by weight, of sucrose palmitate, the percentages by weight in each case relating to calcium valproate employed.

8. Pharmaceutical composition according to one of claims 1 to 7, characterized in that it is present as a divisible tablet.

9. Pharmaceutical composition according to claim 8, characterized in that the tablet or a part thereof obtainable after division has a delayed release of active compound of at most 70% after 4 hours and complete release after 12-16 hours in vitro at pH 6.8, in each case based on the active compound content.

10. Process for the preparation of a pharmaceutical composition comprising calcium valproate with delayed release of active compound according to one of claims 1 to 9, characterized by the steps of introduction into calcium valproate of an aqueous dispersion comprising Eudragit® NE or Eudragit® RS or mixtures thereof, an aqueous dispersion comprising Eudragit® L or Eudragit® S or shellac or mixtures thereof, and an aqueous solution of a sugar ester, if appropriate introduction of suitable excipients, such as talc, microcrystalline cellulose and magnesium stearate, and if appropriate one or more drying steps, a tabletting mixture being obtained which is then compressed to give tablets.

11. Process according to claim 10, characterized in that the introduction into calcium valproate takes place in the sequence 1.) Eudragit® NE or Eudragit® RS or mixtures thereof, 2.) Eudragit® L or Eudragit® S or shellac or mixtures thereof and 3.) sugar ester, in each case followed by drying steps.

12. Process according to claim 10 or 11, characterized in that the aqueous dispersion comprising Eudragit® L or Eudragit® S or shellac or mixtures thereof additionally contains talc.

13. Process according to one of claims 10 to 12, characterized in that the sugar ester used has an HLB of greater than 10, preferably of 14 to 16, particularly preferably of 15 to 16, and a water solubility at 37° C. of no more than 1 part of sugar ester to 100 parts of water, and a water solubility at 60-80° C. of at least 1 part of sugar ester to 10 parts of water, in each case based on the weight.

14. Pharmaceutical composition with delayed release of active compound, characterized in that it is obtainable by the process according to one of claims 10 to 13.

15. Use of the pharmaceutical compositions according to one of claims 1 to 9 and 14 for the treatment of epilepsy in humans and other mammals.

16. Use according to claim 15, characterized in that the treatment of epilepsy is carried out by peroral administration one to two times daily.

17. Use of the pharmaceutical compositions according to one of claims 1 to 9 and 14 for the treatment of migraine in humans and other mammals.

18. Use of the pharmaceutical compositions according to one of claims 1 to 9 and 14 for the treatment of manic-depressive conditions in humans and other mammals.
